(19) United States
(12) Patent Application Publication
(10) Pub. No.: US 2026/0053752 A1
(43) Pub. Date: Feb. 26, 2026

(54) Thermo-Sensitive Electrolyte Supplement with Delayed Release for Enhanced Hydration

(71) Applicant: Caitlin Ferzli, New York, NY (US)

(72) Inventor: Caitlin Ferzli, New York, NY (US)

(21) Appl. No.: 18/813,031

(22) Filed: Aug. 23, 2024

(51) Int. Cl. A61K 9/50; A61K 33/06

(52) U.S. Cl. CPC A61K 9/5047; A61K 9/5026; A61K 33/06

(57) ABSTRACT

A thermo-sensitive electrolyte supplement designed to release electrolytes in response to elevated body temperatures. The supplement, available in capsules, powders, and gummies, uses polymers like polyvinyl alcohol (PVA) and methylcellulose to encapsulate the electrolytes, ensuring controlled and gradual release during physical activity or heat exposure.

### BACKGROUND

This invention relates to dietary supplements, specifically to an electrolyte supplement designed to release electrolytes in response to elevated body temperatures.

Current electrolyte supplements typically release their contents immediately upon ingestion. There is a need for a supplement that releases electrolytes gradually in response to a human's body temperature increase, thereby providing sustained hydration and preventing electrolyte imbalance during physical activity or heat exposure.

### SUMMARY OF INVENTION

The present invention provides a thermo-sensitive electrolyte supplement designed to release electrolytes in response to elevated body temperatures. The supplement can be in the form of capsules, powders combined with water, or gummies, each containing microencapsulated electrolytes that dissolve or degrade at specific temperatures.

### DETAILED DESCRIPTION

### Materials and Methods

Thermo-Sensitive Polymers: Polymers such as polyvinyl alcohol (PVA) and methylcellulose are selected for their ability to degrade or dissolve at elevated temperatures (37-40° C.).

Electrolyte Composition: The supplement contains essential electrolytes such as sodium, potassium, magnesium, and calcium.

Encapsulation Techniques: Methods like fluidized bed coating, spray drying, and microencapsulation are used to coat the electrolytes with the selected polymers.

### Formulations

Capsules: Electrolyte cores are encapsulated with thermo-sensitive coatings.

Powders: Microencapsulated electrolytes are mixed into a powder that dissolves in water.

Gummies: Gummies are embedded with microencapsulated electrolytes that release when the body temperature increases.

Release Mechanism: The coating materials are engineered to dissolve or degrade at specific temperature ranges (37-40° C.), ensuring a controlled and gradual release of electrolytes as the body temperature rises.

Benefits: The thermo-sensitive release mechanism ensures that electrolytes are provided precisely when needed, enhancing hydration and electrolyte balance during physical exertion or exposure to heat.

Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A thermo-sensitive electrolyte supplement comprising microencapsulated electrolytes, wherein the encapsulating material is a polymer selected from the group consisting of polyvinyl alcohol (PVA) and methylcellulose, designed to degrade at temperatures between 37-40°C.

2. The supplement of claim 1, wherein the electrolytes include sodium, potassium, magnesium, and calcium.

3. The supplement of claim 1, in the form of a capsule.

4. The supplement of claim 1, in the form of a powder for dissolution in water.

5. The supplement of claim 1, in the form of a gummy.
